(19) United States
(12) Patent Application Publication
(10) Pub. No.: US 2002/0169302 A1
(43) Pub. Date: Nov. 14, 2002

(54) Compositions isolated from bovine mammary gland and methods for their use

(75) Inventors: Illka J. Havukkala, Auckland (NZ); Matthew Glenn, Auckland (NZ); Murray R. Grigor, Auckland (NZ); Adrian J. Molenaar, Hamilton (NZ)

Correspondence Address: SPECHMAN LAW GROUP, 1501 WESTERN AVE, SUITE 100, SEATTLE, WA, 98101, US

(73) Assignee: Genesis Research and Development Corp. Ltd., Auckland (NZ)

(21) Appl. No.: 10/079,623

(22) Filed: Feb. 19, 2002

Related U.S. Application Data
(63) Continuation-in-part of application No. 09/644,199, filed on Aug. 22, 2000.
(60) Provisional application No. 60/150,330, filed on Aug. 23, 1999.

(51) Int. Cl. C07H 21/02; C07H 21/04

(52) U.S. Cl. 536/23.1

(57) ABSTRACT

Isolated polynucleotides encoding polypeptides expressed in bovine mammary gland tissue are provided, together with genetic constructs and host cells comprising such isolated polynucleotides. Methods for the use of such polynucleotides and polypeptides are also provided.

### CROSS-REFERENCE TO RELATED APPLICATIONS

This application is a continuation-in-part of U.S. patent application Ser. No. 09/644,190, filed Aug. 22, 2000, which claims priority to U.S. Provisional Patent Application No. 60/150,330, filed Aug. 23, 1999.

### TECHNICAL FIELD OF THE INVENTION

This invention relates to polypeptides expressed in bovine mammary gland cells, to polynucleotides encoding such polypeptides, and to methods for treating a mammal involving administration of a polypeptide or polynucleotide of the present invention.

### BACKGROUND OF THE INVENTION

The bovine mammary gland is a milk-producing organ of great economic importance. Knowledge of the genes expressed in this tissue is valuable in understanding the physiology and function of the mammary gland, not only in the cow, but also in other mammals, including humans. The polynucleotide sequences themselves are useful in detecting the presence of mammary gland tissue in a biological sample, and in the development of transgenic animals with transgene expression targeted to the mammary gland, e.g., by using mammary gland-specific promoters isolated from mammary-specific polynucleotides.

### SUMMARY OF THE INVENTION

The present invention provides isolated polypeptides expressed in bovine mammary gland cells and isolated polynucleotides encoding such polypeptides, together with genetic constructs and host cells comprising such polynucleotides. Methods for using such polypeptides, polynucleotides and genetic constructs are also provided.

In specific embodiments, isolated polynucleotides are provided that comprise a polynucleotide sequence selected from the group consisting of: (a) sequences recited in SEQ ID NOS: 1-348; (b) complements of the sequences recited in SEQ ID NOS: 1-348; (c) reverse complements of the sequences recited in SEQ ID NOS: 1-348; (d) reverse sequences of the sequences recited in SEQ ID NOS: 1-348; (e) sequences having a 99% probability of being to the same as a sequence of (a)-(d), determined as described below; and (f) sequences having at least 50%, 75%, 90% or 95% identity to a sequence of (a)-(d), the percentage identity being determined as described below. Polynucleotides comprising at least a specified number of contiguous residues (“x-mers”) of any of the sequences identified as SEQ ID NOS: 1-348 are also provided, together with extended sequences, and oligonucleotide probes and primers corresponding to the sequences set out in SEQ ID NOS: 1-348. All of these polynucleotides and oligonucleotide probes and primers are collectively referred to herein as “polynucleotides of the present invention”.

In further embodiments, the present invention provides isolated polypeptides comprising an amino acid sequence encoded by a polynucleotide selected from the group consisting of: (a) sequences provided in SEQ ID NOS: 1-348; and (b) sequences having at least 50%, 75%, 90% or 95% identity to a sequence provided in SEQ ID NOS: 1-348, together with isolated polynucleotides encoding such polypeptides. Isolated polypeptides comprising at least a functional portion of an amino acid sequence encoded by a polynucleotide selected from the group consisting of: (a) sequences provided in SEQ ID NOS: 1-348; and (b) sequences having 50%, 75%, 90% or 95% identity to a sequence of SEQ ID NOS: 1-348, are also provided. In specific embodiments, such polypeptides comprise a sequence selected from the group consisting of sequences identified as SEQ ID NO: 349-370, and variants thereof.

In related embodiments, the present invention provides genetic constructs comprising the above polynucleotides, together with host cells transformed with such constructs, and organisms comprising such host cells.

In a further aspect, the present invention provides methods for stimulating bovine mammary gland cell growth and function, inhibiting the growth of various mammary gland cancer cells, inhibiting angiogenesis and vascularization of tumors, or modulating the growth of blood vessels in a mammal, such methods comprising administering to the subject a composition comprising an isolated polypeptide of the present invention. Methods for modulating mammary gland function in a mammal are also provided, the methods comprising administering to the subject a composition comprising an inventive polypeptide.

As detailed below, the isolated polynucleotides and polypeptides of the present invention may be usefully employed in the preparation of therapeutic agents for the treatment of mammary gland disorders. In addition, polynucleotides that are specifically expressed at a higher or lower level in diseased mammary gland than in a normal mammary gland may be used as an indicator of the disease condition. Similarly, disposition to a disease related to a specific level of expression of a polynucleotide would indicate use of that polynucleotide as a marker for diagnosis of susceptible individuals. In yet another aspect, the mapping of a specific polynucleotide of this invention close to the chromosomal location of any beneficial or detrimental genes would make the polynucleotide a valuable tool for breeding of livestock, disease diagnostics, or identification of the beneficial or detrimental gene.

The isolated polynucleotides of the present invention have further utility in genome mapping, in physical mapping, and in positional cloning of genes. Additionally, the polynucleotide sequences identified as SEQ ID NOS: 1-348, and their variants, may be used to design oligonucleotide probes and primers (referred to collectively as “oligonucleotides”). As detailed below, oligonucleotide probes and primers have sequences that are substantially complementary to the polynucleotide of interest over a certain portion of the polynucleotide. The inventive oligonucleotide probes may be used to detect the presence, and examine the expression patterns, of genes in any organism having sufficiently similar DNA and RNA sequences in their cells using techniques that are well known in the art, such as slot blot DNA hybridization techniques. The inventive oligonucleotide primers may be used for PCR amplifications. Oligonucleotide probes and primers of the present invention may also be used in connection with various microarray technologies, including the microarray technology of Affymetrix, Inc. (Santa Clara, Calif.).

The above-mentioned and additional features of the present invention, together with the manner of obtaining them, will be best understood by reference to the following more detailed description. All references disclosed herein are incorporated herein by reference in their entirety as if each was incorporated individually.

### DETAILED DESCRIPTION OF THE INVENTION

In certain aspects, the present invention provides polynucleotides that were isolated by sequencing of cDNA libraries from bovine mammary gland cells, together with isolated polypeptides encoded by such polynucleotides.

The polynucleotides of the present invention encode polypeptides that are present in bovine mammary gland tissue and that may therefore have important roles in growth, development and function of mammary gland cells, and in responses of mammary gland cells to tissue injury and inflammation, as well as disease states, and which have antibacterial or other bioactive utility. The polypeptides and/or polynucleotides of the present invention may thus be employed in the modification of mammary function, as potential markers for selection of livestock having enhanced mammary performance, and as diagnostics for abnormal cellular growth in mammary cancer. Oligonucleotide probes and primers corresponding to the inventive polynucleotides may be employed to detect the presence of mammary gland tissue in a specific tissue sample using techniques well known in the art, such as DNA hybridization and polymerase chain reaction (PCR) amplification.

The inventive polypeptides have important roles in processes such as induction of mammary growth, differentiation of milk producing cells, cell migration, cell proliferation, and cell-cell interaction. The polypeptides are important in the maintenance of tissue integrity, and thus are important in processes such as wound healing. Some of the disclosed polypeptides can act as modulators of immune responses, especially since milk is known to contain immunologically active polypeptides for the benefit of mammal offspring. In addition, many polypeptides are immunologically active also within the mammary gland, making them important therapeutic targets in a whole range of disease states not only within the mammary gland, but also in other tissues of a mammal. Antibodies to the polypeptides of the present invention and small molecule inhibitors related to the polypeptides of the present invention may also be used for modulating immune responses and for treatment of diseases according to the present invention.

Isolated polynucleotides of the present invention include the polynucleotides identified herein as SEQ ID NOS: 1-348; isolated polynucleotides comprising a polynucleotide sequence selected from the group consisting of SEQ ID NOS: 1-348; isolated polynucleotides comprising at least a specified number of contiguous residues (x-mers) of any of the polynucleotides identified as SEQ ID NOS: 1-348; isolated polynucleotides comprising a polynucleotide sequence that is complementary to any of the above polynucleotides; isolated polynucleotides comprising a polynucleotide sequence that is a reverse sequence or a reverse complement of any of the above polynucleotides; antisense sequences corresponding to any of the above polynucleotides; and variants of any of the above polynucleotides, as that term is described in this specification.

The definition of the terms “complement,” “reverse complement,” and “reverse sequence,” as used herein, is best illustrated by the following example. For the sequence 5′ AGGACC 3′, the complement, reverse complement, and reverse sequence are as follows:

| | |
|---|---|
| complement | 3′ TCCTGG 5′ |
| reverse complement | 3′ GGTCCT 5′ |
| reverse sequence | 5′ CCAGGA 3′. |

Preferably, sequences that are complements of a specifically recited polynucleotide sequence are complementary over the entire length of the specific polynucleotide sequence.

As used herein, the term “oligonucleotide” refers to a relatively short segment of a polynucleotide sequence, generally comprising between 6 and 60 nucleotides, and comprehends both probes for use in hybridization assays and primers for use in the amplification of DNA by polymerase chain reaction.

As used herein, the term “polynucleotide” means a single- or double-stranded polymer of deoxyribonucleotide or ribonucleotide bases and includes DNA and RNA molecules, both sense and anti-sense strands. The term comprehends cDNA, genomic DNA, recombinant DNA, and wholly or partially synthesized nucleic acid molecules. A polynucleotide may consist of an entire gene, or a portion thereof. A gene is a DNA sequence that codes for a functional protein or RNA molecule. Operable anti-sense polynucleotides may comprise a fragment of the corresponding polynucleotide, and the definition of “polynucleotide” therefore includes all operable anti-sense fragments. Anti-sense polynucleotides and techniques involving anti-sense polynucleotides are well known in the art and are described, for example, in Robinson-Benion et al., “Anti-sense techniques,” Methods in Enzymol. 254(23):363-375, 1995; and Kawasaki et al., Artific. Organs 20(8):836-848, 1996.

Identification of genomic DNA and heterologous species DNA can be accomplished by standard DNA/DNA hybridization techniques, under appropriately stringent conditions, using all or part of a polynucleotide sequence as a probe to screen an appropriate library. Alternatively, PCR techniques using oligonucleotide primers that are designed based on known genomic DNA, cDNA and protein sequences can be used to amplify and identify genomic and/or cDNA sequences. Synthetic polynucleotides corresponding to the identified sequences, and variants thereof, may be produced by conventional synthesis methods.

All the polynucleotides and polypeptides provided by the present invention are isolated and purified, as those terms are commonly used in the art. Preferably, the inventive polypeptides and polynucleotides are at least about 80% pure, more preferably at least about 90% pure, and most preferably at least about 99% pure.

The polynucleotide sequences identified as SEQ ID NOS: 1-348 were derived from bovine mammary gland cells. Certain of the polynucleotides of the present invention may be “partial” sequences, in that they do not represent a full-length gene encoding a full-length polypeptide. Such partial sequences may be extended by analyzing and sequencing various DNA libraries using primers and/or probes and well known hybridization and/or PCR techniques. Polynucleotide sequences disclosed herein may thus be extended until an open reading frame encoding a polypeptide, a full-length polynucleotide and/or gene capable of expressing a polypeptide, or another useful portion of the genome is identified. Such extended sequences, including full length polynucleotides and genes, are described as “corresponding to” a sequence identified as one of the sequences of SEQ ID NOS: 1-348, or a variant thereof, or a portion of one of the sequences of SEQ ID NOS: 1-348, or a variant thereof, when the extended polynucleotide comprises an identified sequence or its variant, or an identified contiguous portion (x-mer) of one of the sequences of SEQ ID NOS: 1-348 or a variant thereof.

The polynucleotides identified as SEQ ID NOS: 1-348 were isolated from bovine mammary gland cDNA libraries and represent sequences that are expressed in the tissue from which the cDNA was prepared. The sequence information may be used to isolate or synthesize expressible DNA molecules, such as open reading frames or full-length genes, that can then be used as expressible or otherwise functional DNA in cows and other organisms. Similarly, RNA sequences, reverse sequences, complementary sequences, antisense sequences and the like, corresponding to the polynucleotides of the present invention, may be routinely ascertained and obtained using the cDNA sequences identified as SEQ ID NOS: 1-348.

The polynucleotides identified as SEQ ID NOS: 1-348 may contain open reading frames (“ORFs”) or partial open reading frames encoding polypeptides. Additionally, open reading frames encoding polypeptides may be identified in extended or full-length sequences corresponding to the sequences set out as SEQ ID NOS: 1-348. Open reading frames may be identified using techniques that are well known in the art. These techniques include, for example, analysis for the location of known start and stop codons, most likely reading frame identification based on codon frequencies, etc. Suitable tools and software for ORF analysis are available, for example, on the Internet at the National Institutes of Health NCBI website. Additional tools and software for ORF analysis are available, and include GeneWise, available from The Sanger Center, Wellcome Trust Genome Campus, Hinxton, Cambridge, CB10 1SA, United Kingdom; Diogenes, available from Computational Biology Centers, University of Minnesota, Academic Health Center, UMHG Box 43 Minneapolis Minn. 55455; and GRAIL, available from the Informatics Group, Oak Ridge National Laboratories, Oak Ridge, Tennessee Tenn. Open reading frames and portions of open reading frames may be identified in the polynucleotides of the present invention. Once a partial open reading frame is identified, the polynucleotide may be extended in the area of the partial open reading frame using techniques that are well known in the art until the polynucleotide for the full open reading frame is identified. Thus, polynucleotides and open reading frames encoding polypeptides may be identified using the polynucleotides of the present invention.

Once open reading frames are identified in the polynucleotides of the present invention, the open reading frames may be isolated and/or synthesized. Expressible genetic constructs comprising the open reading frames and suitable promoters, initiators, terminators, etc., which are well known in the art, may then be constructed. Such genetic constructs may be introduced into a host cell to express the polypeptide encoded by the open reading frame. Suitable host cells may include various prokaryotic and eukaryotic cells, including mammalian cells. In vitro expression of polypeptides is also possible, as well known in the art.

Polypeptides encoded by the polynucleotides of the present invention may be expressed and used in various assays to determine their biological activity. Such polypeptides may be used to raise antibodies, to isolate corresponding interacting proteins or other compounds, and to quantitatively determine levels of interacting proteins or other compounds.

In another aspect, the present invention provides isolated polypeptides encoded, or partially encoded, by the above polynucleotides. As used herein, the term “polypeptide” encompasses amino acid chains of any length, including full-length proteins, wherein the amino acid residues are linked by covalent peptide bonds. The term “polypeptide encoded by a polynucleotide” as used herein, includes polypeptides encoded by a polynucleotide that comprises an isolated polynucleotide sequence or variant provided herein. Polypeptides of the present invention may be naturally purified products, or may be produced partially or wholly using recombinant techniques. Such polypeptides may be glycosylated with bacterial, fungal, mammalian or other eukaryotic carbohydrates or may be non-glycosylated. In specific embodiments, the inventive polypeptides comprise an amino acid sequence encoded by a polynucleotide selected from the group consisting of sequences provided in SEQ ID NOS: 1-348, including the amino acid sequences identified as SEQ ID NO: 349-370, as well as variants of such sequences.

Polypeptides of the present invention may be produced recombinantly by inserting a polynucleotide sequence that encodes the polypeptide into a genetic construct and expressing the polypeptide in an appropriate host. Any of a variety of genetic constructs known to those of ordinary skill in the art may be employed. Expression may be achieved in any appropriate host cell that has been transformed or transfected with a genetic construct containing a polynucleotide that encodes a recombinant polypeptide. Suitable host cells include prokaryotes, yeast, and higher eukaryotic cells. Preferably, the host cells employed are Escherichia coli, insect, yeast, or a mammalian cell line such as COS or CHO. The polynucleotide sequences expressed in this manner may encode naturally occurring polypeptides, portions of naturally occurring polypeptides, or other variants thereof.

In a related aspect, polypeptides are provided that comprise at least a functional portion of a polypeptide having an amino acid sequence encoded by a polynucleotide of the present invention. As used herein, the “functional portion” of a polypeptide is that portion which contains the active site essential for affecting the function of the polypeptide, for example, the portion of the molecule that is capable of binding one or more reactants. The active site may be made up of separate portions present on one or more polypeptide chains and will generally exhibit high binding affinity.

Functional portions of a polypeptide may be identified by first preparing fragments of the polypeptide by either chemical or enzymatic digestion of the polypeptide, or by mutation analysis of the polynucleotide that encodes the polypeptide and subsequent expression of the resulting mutant polypeptides. The polypeptide fragments or mutant polypeptides are then tested to determine which portions retain biological activity, using, for example, the representative assays provided below.

Portions and other variants of the inventive polypeptides may also be generated by synthetic or recombinant means. Synthetic polypeptides having fewer than about 100 amino acids, and generally fewer than about 50 amino acids, may be generated using techniques well known to those of ordinary skill in the art. For example, such polypeptides may be synthesized using any of the commercially available solid-phase techniques, such as the Merrifield solid-phase synthesis method, where amino acids are sequentially added to a growing amino acid chain. See Merrifield, J. Am. Chem. Soc. 85:2149-2154, 1963. Equipment for automated synthesis of polypeptides is commercially available from suppliers such as Perkin Elmer/Applied BioSystems, Inc. (Foster City, Calif.), and may be operated according to the manufacturer's instructions. Variants of a native polypeptide may be prepared using standard mutagenesis techniques, such as oligonucleotide-directed, site-specific mutagenesis (Kunkel, Proc. Natl. Acad. Sci. USA 82:488-492, 1985). Sections of polynucleotide sequence may also be removed using standard techniques to permit preparation of truncated polypeptides.

As used herein, the term “variant” comprehends nucleotide or amino acid sequences different from the specifically identified sequences, wherein one or more nucleotides or amino acid residues is deleted, substituted, or added. Variants may be naturally occurring allelic variants, or non-naturally occurring variants. Variant sequences (polynucleotide or polypeptide) preferably exhibit at least 50%, more preferably at least 75%, more preferably yet at least 90%, and most preferably at least 95% identity to a sequence of the present invention. The percentage identity is determined by aligning the two sequences to be compared as described below, determining the number of identical residues in the aligned portion, dividing that number by the total number of residues in the inventive (queried) sequence, and multiplying the result by 100. By way of example only, assume a queried polynucleotide having 220 nucleic acids has a hit to a polynucleotide sequence in the EMBL database having 520 nucleic acids over a stretch of 23 nucleotides in the alignment produced by the BLASTN algorithm using the default parameters as described below. The 23 nucleotide hit includes 21 identical nucleotides, one gap and one different nucleotide. The percentage identity of the queried polynucleotide to the hit in the EMBL database is thus 21/220 times 100, or 9.5%. The percentage identity of polypeptide sequences may be determined in a similar fashion.

Polynucleotide and polypeptide sequences may be aligned, and percentages of identical residues in a specified region may be determined against another polynucleotide or polypeptide sequence, using computer algorithms that are publicly available. Two exemplary algorithms for aligning and identifying the similarity of polynucleotide sequences are the BLASTN and FASTA algorithms. Polynucleotides may also be analyzed using the BLASTX algorithm, which compares the six-frame conceptual translation products of a nucleotide query sequence (both strands) against a protein sequence database. The percentage identity of polypeptide sequences may be examined using the BLASTP algorithm. The BLASTN, BLASTP and BLASTX algorithms are available on the NCBI anonymous FTP server under Iblast/executables/ and are available from the National Center for Biotechnology Information (NCBI), National Library of Medicine, Building 38A, Room 8N805, Bethesda, Md. 20894, USA. The BLASTN algorithm Version 2.0.4 [Feb. 24, 1998], Version 2.0.6 [Sep. 16, 1998] and Version 2.0.11 [Jan. 20, 2000], set to the parameters described below, is preferred for use in the determination of polynucleotide variants according to the present invention. The BLASTP algorithm, set to the parameters described below, is preferred for use in the determination of polypeptide variants according to the present invention. The use of the BLAST family of algorithms, including BLASTN, BLASTP and BLASTX, is described in the publication of Altschul, et al., Nucleic Acids Res. 25: 3389-3402, 1997.

The FASTA and FASTX algorithms are available on the Internet, and from the University of Virginia by contacting the Vice Provost for Research, University of Virginia, P.O. Box 9025, Charlottesville, Va. 22906-9025, USA. The FASTA algorithm, set to the default parameters described in the documentation and distributed with the algorithm, may be used in the determination of polynucleotide variants. The readme files for FASTA and FASTX Version 1.Ox that are distributed with the algorithms describe the use of the algorithms and describe the default parameters. The use of the FASTA and FASTX algorithms is described in Pearson and Lipman, Proc. NatL. Acad. Sci. USA 85:2444-2448, 1988; and Pearson, Methods in Enzymol. 183:63-98, 1990.

The following running parameters are preferred for determination of alignments and similarities using BLASTN that contribute to the E values and percentage identity for polynucleotides: Unix running command with the following default parameters: blastall −p blastn −d embldb −e 10 −G 0 −E 0 −r 1 −v 30 −b 30 −i queryseq −o results; and parameters are: −p Program Name [String]; −d Database [String]; −e Expectation value (E) [Real]; −G Cost to open a gap (zero invokes default behavior) [Integer]; −E Cost to extend a gap (zero invokes default behavior) [Integer]; −r Reward for a nucleotide match (blastn only) [Integer]; −v Number of one-line descriptions (V) [Integer]; −b Number of alignments to show (B) [Integer]; −i Query File [File In]; −o BLAST report Output File [File Out] Optional.

The following running parameters are preferred for determination of alignments and similarities using BLASTP that contribute to the E values and percentage identity of polypeptide sequences: blastall −p blastp −d swissprotdb −e 10 −G 0 −E 0 −v 30 −b 30 −i queryseq −o results; the parameters are: −p Program Name [String]; −d Database [String]; −e Expectation value (E) [Real]; −G Cost to open a gap (zero invokes default behavior) [Integer]; −E Cost to extend a gap (zero invokes default behavior) [Integer]; −v Number of one-line descriptions (v) [Integer]; −b Number of alignments to show (b) [Integer]; −I Query File [File In]; −o BLAST report Output File [File Out] Optional.

The “hits” to one or more database sequences by a queried sequence produced by BLASTN, BLASTP, FASTA, or a similar algorithm, align and identify similar portions of sequences. The hits are arranged in order of the degree of similarity and the length of sequence overlap. Hits to a database sequence generally represent an overlap over only a fraction of the sequence length of the queried sequence. The BLASTN, FASTA and BLASTP algorithms also produce “Expect” values for polynucleotide and polypeptide alignments. The Expect value (E) indicates the number of hits one can “expect” to see over a certain number of contiguous sequences by chance when searching a database of a certain size. The Expect value is used as a significance threshold for determining whether the hit to a database indicates true similarity. For example, an E value of 0.1 assigned to a polynucleotide hit is interpreted as meaning that in a database of the size of the EMBL database, one might expect to see 0.1 matches over the aligned portion of the sequence with a similar score simply by chance. By this criterion, the aligned and matched portions of the sequences then have a probability of 90% of being related. For sequences having an E value of 0.01 or less over aligned and matched portions, the probability of finding a match by chance in the EMBL database is 1% or less using the BLASTN algorithm. E values for polypeptide sequences may be determined in a similar fashion using various polypeptide databases, such as the SwissProt database.

According to one embodiment, “variant” polynucleotides and polypeptides, with reference to each of the polynucleotides and polypeptides of the present invention, preferably comprise sequences having the same number or fewer nucleic or amino acids than each of the polynucleotides or polypeptides of the present invention and producing an E value of 0.01 or less when compared to the polynucleotide or polypeptide of the present invention. That is, a variant polynucleotide or polypeptide is any sequence that has at least a 99% probability of being the same as the polynucleotide or polypeptide of the present invention, measured as having an E value of 0.01 or less using the BLASTN, FASTA or BLASTP algorithms set at the default parameters. According to a preferred embodiment, a variant polynucleotide is a sequence having the same number or fewer nucleic acids than a polynucleotide of the present invention that has at least a 99% probability of being the same as the polynucleotide of the present invention, measured as having an E value of 0.01 or less using the BLASTN algorithm set at the default parameters. Similarly, according to a preferred embodiment, a variant polypeptide is a sequence having the same number or fewer amino acids than a polypeptide of the present invention that has at least a 99% probability of being the same as the polypeptide of the present invention, measured as having an E value of 0.01 or less using the BLASTP algorithm set at the default parameters.

In addition to having a specified percentage identity to an inventive polynucleotide or polypeptide sequence, variant polynucleotides and polypeptides preferably have additional structure and/or functional features in common with the inventive polynucleotide or polypeptide. Polypeptides having a specified degree of identity to a polypeptide of the present invention share a high degree of similarity in their primary structure and have substantially similar functional properties. In addition to sharing a high degree of similarity in their primary structure to polynucleotides of the present invention, polynucleotides having a specified degree of identity to, or capable of hybridizing to, an inventive polynucleotide preferably have at least one of the following features: (i) they contain an open reading frame or partial open reading frame encoding a polypeptide having substantially the same functional properties as the polypeptide encoded by the inventive polynucleotide; or (ii) they contain identifiable domains in common.

In certain embodiments, variant polynucleotides hybridize to a polynucleotide of the present invention under stringent conditions. As used herein, “stringent conditions” refers to prewashing in a solution of 6× SSC, 0.2% SDS; hybridizing at 65° C., 6× X SSC, 0.2% SDS overnight; followed by two washes of 30 minutes each in 1× X SSC, 0.1% SDS at 65° C. and two washes of 30 minutes each in 0.2× X SSC, 0.1% SDS at 65° C.

The present invention also encompasses polynucleotides that differ from the disclosed sequences but that, as a consequence of the discrepancy of the genetic code, encode a polypeptide having similar enzymatic activity as a polypeptide encoded by a polynucleotide of the present invention. Thus, polynucleotides comprising sequences that differ from the polynucleotide sequences recited in SEQ ID NOS: 1-348 (or complements, reverse sequences, or reverse complements of those sequences) as a result of conservative substitutions are encompassed within the present invention. Additionally, polynucleotides comprising sequences that differ from the inventive polynucleotide sequences or complements, reverse complements, or reverse sequences as a result of deletions and/or insertions totaling less than 10% of the total sequence length are also contemplated by and encompassed within the present invention. Similarly, polypeptides comprising sequences that differ from the inventive polypeptide sequences as a result of amino acid substitutions, insertions, and/or deletions totaling less than 10% of the total sequence length are contemplated by and encompassed within the present invention, provided the variant polypeptide has similar activity to the inventive polypeptide.

The polynucleotides of the present invention may be isolated from bovine cDNA libraries as described below, or may be synthesized using techniques that are well known in the art. The polynucleotides may be synthesized, for example, using automated oligonucleotide synthesizers (e.g., Beckman Oligo 1000M DNA Synthesizer) to obtain polynucleotide segments of up to 50 or more nucleic acids. A plurality of such polynucleotide segments may then be ligated using standard DNA manipulation techniques that are well known in the art of molecular biology. One conventional and exemplary polynucleotide synthesis technique involves synthesis of a single stranded polynucleotide segment having, for example, 80 nucleic acids, and hybridizing that segment to a synthesized complementary 85 nucleic acid segment to produce a 5 nucleotide overhang. The next segment may then be synthesized in a similar fashion, with a 5 nucleotide overhang on the opposite strand. The “sticky” ends ensure proper ligation when the two portions are hybridized. In this way, a complete polynucleotide of the present invention may be synthesized entirely in vitro.

As noted above, certain of the polynucleotides identified as SEQ ID NOS: 1-348 may be referred to as “partial” sequences, in that they may not represent the full coding portion of a gene encoding a naturally occurring polypeptide. Partial polynucleotide sequences disclosed herein may be employed to obtain the corresponding full-length genes for various species and organisms by, for example, screening DNA expression libraries using hybridization probes based on the polynucleotides of the present invention, or using PCR amplification with primers based upon the polynucleotides of the present invention. In this way one can, using methods well known in the art, extend a polynucleotide of the present invention upstream and downstream of the corresponding mRNA, as well as identify the corresponding genomic DNA, including the promoter and enhancer regions, of the complete gene. The present invention thus comprehends isolated polynucleotides comprising a sequence identified in SEQ ID NOS: 1-348, or a variant of one of the specified sequences, that encode a functional polypeptide, including full-length genes. Such extended polynucleotides may have a length of from about 50 to about 4,000 nucleic acids or base pairs, and preferably have a length of less than about 4,000 nucleic acids or base pairs, more preferably yet a length of less than about 3,000 nucleic acids or base pairs, more preferably yet a length of less than about 2,000 nucleic acids or base pairs. Under some circumstances, extended polynucleotides of the present invention may have a length of less than about 1,800 nucleic acids or base pairs, preferably less than about 1,600 nucleic acids or base pairs, more preferably less than about 1,400 nucleic acids or base pairs, more preferably yet less than about 1,200 nucleic acids or base pairs, and most preferably less than about 1,000 nucleic acids or base pairs.

As used herein, the term “x-mer,” with reference to a specific value of “x,” refers to a polynucleotide or polypeptide, respectively, comprising at least a specified number (“x”) of contiguous residues of: any of the polynucleotides provided in SEQ ID NOS: 1-348. The value of x may be from about 20 to about 600, depending upon the specific sequence.

Polynucleotides of the present invention comprehend polynucleotides comprising at least a specified number of contiguous residues (x-mers) of any of the polynucleotides identified as SEQ ID NOS: 1-348, or their variants. Polypeptides of the present invention comprehend polypeptides comprising at least a specified number of contiguous residues (x-mers) of any of the polypeptides corresponding to the polynucleotides of SEQ ID NOS: 1-348. According to preferred embodiments, the value of x is at least 20, more preferably at least 40, more preferably yet at least 60, and most preferably at least 80. Thus, polynucleotides of the present invention include polynucleotides comprising a 20-mer, a 40-mer, a 60-mer, an 80-mer, a 100-mer, a 120-mer, a 150-mer, a 180-mer, a 220-mer, a 250-mer, a 300-mer, 400-mer, 500-mer or 600-mer of a polynucleotide provided in SEQ ID NOS: 1-348, or a variant of one of the polynucleotides provided in SEQ ID NOS: 1-348. Similarly, polypeptides of the present invention include polypeptides comprising a 20-mer, a 40-mer, a 60-mer, an 80-mer, a 100-mer, a 120-mer, a 150-mer, a 180-mer, a 220-mer, a 250-mer, a 300-mer, 400-mer, 500-mer or 600-mer of a polypeptide encoded by a polynucleotide provided in SEQ ID NOS: 1-348, or a variant of one of the polynucleotides provided in SEQ ID NOS: 1-348.

The inventive polynucleotides may be isolated by high throughput sequencing of cDNA libraries prepared from bovine mammary gland tissue as described below in Example 1. Alternatively, oligonucleotide probes and/or primers based on the sequences provided in SEQ ID NOS: 1-348, can be synthesized and used to identify positive clones in either cDNA or genomic DNA libraries from bovine mammary gland cells by means of hybridization or polymerase chain reaction (PCR) techniques. Probes can be shorter than the sequences provided herein but should be at least about 10, preferably at least about 15 and most preferably at least about 20 nucleotides in length. Hybridization and PCR techniques suitable for use with such oligonucleotide probes are well known in the art (see, for example, Mullis et al., Cold Spring Harbor Symp. Quant. Biol., 51:263, 1987; Erlich, ed., PCR technology, Stockton Press: N.Y., 1989; and Sambrook et al., in Molecular cloning: a laboratory manual, 2nd ed., CSHL Press: Cold Spring Harbor, N.Y., 1989). Positive clones may be analyzed by restriction enzyme digestion, DNA sequencing or the like.

In addition, polynucleotide sequences of the present invention may be generated by synthetic means using techniques well known in the art. Equipment for automated synthesis of oligonucleotides is commercially available from suppliers such as Perkin Elmer/Applied Biosystems Division (Foster City, Calif.) and may be operated according to the manufacturer's instructions.

Oligonucleotide probes and primers complementary to and/or corresponding to SEQ ID NOS: 1-348, and variants of those sequences, are also comprehended by the present invention. Such oligonucleotide probes and primers are substantially complementary to the polynucleotide of interest. An oligonucleotide probe or primer is described as “corresponding to” a polynucleotide of the present invention, including one of the sequences set out as SEQ ID NOS: 1-348 or a variant thereof, if the oligonucleotide probe or primer, or its complement, is contained within one of the sequences set out as SEQ ID NOS: 1-348 or a variant of one of the specified sequences.

Two single stranded sequences are said to be substantially complementary when the nucleotides of one strand, optimally aligned and compared, with the appropriate nucleotide insertions and/or deletions, pair with at least 80%, preferably at least 90% to 95%, and more preferably at least 98% to 100%, of the nucleotides of the other strand. Alternatively, substantial complementarity exists when a first DNA strand will selectively hybridize to a second DNA strand under stringent hybridization conditions. Stringent hybridization conditions for determining complementarity include salt conditions of less than about 1 M, more usually less than about 500 mM, and preferably less than about 200 mM. Hybridization temperatures can be as low as 5° C., but are generally greater than about 22° C., more preferably greater than about 30° C., and most preferably greater than about 37° C. Longer DNA fragments may require higher hybridization temperatures for specific hybridization. Since the stringency of hybridization may be affected by other factors such as probe composition, presence of organic solvents, and extent of base mismatching, the combination of parameters is more important than the absolute measure of any one alone. DNA-DNA hybridization studies may be performed using either genomic DNA or DNA derived by preparing cDNA from the RNA present in the sample.

In addition to DNA-DNA hybridization, DNA-RNA or RNA-RNA hybridization assays are also possible. In the first case, the mRNA from expressed genes would then be detected instead of genomic DNA or cDNA derived from MRNA of the sample. In the second case, RNA probes could be used. In addition, artificial analogs of DNA hybridizing specifically to target sequences could also be used.

In specific embodiments, the inventive oligonucleotide probes and/or primers comprise at least about 6 contiguous residues, more preferably at least about 10 contiguous residues, and most preferably at least about 20 contiguous residues complementary to a polynucleotide sequence of the present invention. Probes and primers of the present invention may be from about 8 to 100 base pairs in length, or preferably from about 10 to 50 base pairs in length, or more preferably from about 15 to 40 base pairs in length. The probes can be easily selected using procedures well known in the art, taking into account DNA-DNA hybridization stringencies, annealing and melting temperatures, potential for formation of loops, and other factors which are well known in the art. Tools and software suitable for designing probes, and especially suitable for designing PCR primers, are available on the Internet. In addition, a software program suitable for designing probes, and especially for designing PCR primers, is available from Premier Biosoft International, 3786 Corina Way, Palo Alto, Calif. 94303-4504. Preferred techniques for designing PCR primers are also disclosed in Dieffenbach and Dyksler, PCR Primer: a laboratory manual, CSHL Press: Cold Spring Harbor, N.Y., 1995.

A plurality of oligonucleotide probes or primers corresponding to a polynucleotide of the present invention may be provided in a kit form. Such kits generally comprise multiple DNA or oligonucleotide probes, each probe being specific for a polynucleotide sequence. Kits of the present invention may comprise one or more probes or primers corresponding to a polynucleotide of the present invention, including a polynucleotide sequence identified in SEQ ID NOS: 1-348.

In one embodiment useful for high-throughput assays, the oligonucleotide probe kits of the present invention comprise multiple probes in an array format, wherein each probe is immobilized in a predefined, spatially addressable location on the surface of a solid substrate. Array formats which may be usefully employed in the present invention are disclosed, for example, in U.S. Pat. Nos. 5,412,087, 5,545,531, and PCT Publication No. WO 95/00530, the disclosures of which are hereby incorporated by reference.

Oligonucleotide probes for use in the present invention may be constructed synthetically prior to immobilization on an array, using techniques well known in the art (See, for example, Gait, ed., Oligonucleotide synthesis a practical approach, IRL Press: Oxford, England, 1984). Automated equipment for the synthesis of oligonucleotides is available commercially from such companies as Perkin Elmer/Applied Biosystems

Division (Foster City, Calif.) and may be operated according to the manufacturer's instructions. Alternatively, the probes may be constructed directly on the surface of the array using techniques taught, for example, in PCT Publication No. WO 95/00530.

The solid substrate and the surface thereof preferably form a rigid support and are generally formed from the same material. Examples of materials from which the solid substrate may be constructed include polymers, plastics, resins, membranes, polysaccharides, silica or silica-based materials, carbon, metals and inorganic glasses. Synthetically prepared probes may be immobilized on the surface of the solid substrate using techniques well known in the art, such as those disclosed in U.S. Pat. No. 5,412,087.

In one such technique, compounds having protected functional groups, such as thiols protected with photochemically removable protecting groups, are attached to the surface of the substrate. Selected regions of the surface are then irradiated with a light source, preferably a laser, to provide reactive thiol groups. This irradiation step is generally performed using a mask having apertures at predefined locations using photolithographic techniques well known in the art of semiconductors. The reactive thiol groups are then incubated with the oligonucleotide probe to be immobilized. The precise conditions for incubation, such as temperature, time and pH, depend on the specific probe and can be easily determined by one of skill in the art. The surface of the substrate is washed free of unbound probe and the irradiation step is repeated using a second mask having a different pattern of apertures. The surface is subsequently incubated with a second, different, probe. Each oligonucleotide probe is typically immobilized in a discrete area of less than about 1 mm . Preferably each discrete area is less than about 10,000 mm^{2,} more preferably less than about 100 mm². In this manner, a multitude of oligonucleotide probes may be immobilized at predefined locations on the array.

The resulting array may be employed to screen for differences in organisms or samples or products containing genetic material as follows. Genomic or cDNA libraries are prepared using techniques well known in the art. The resulting target DNA is then labeled with a suitable marker, such as a radiolabel, chromophore, fluorophore or chemiluminescent agent, using protocols well known for those skilled in the art. A solution of the labeled target DNA is contacted with the surface of the array and incubated for a suitable period of time.

The surface of the array is then washed free of unbound target DNA and the probes to which the target DNA hybridized are determined by identifying those regions of the array to which the markers are attached. When the marker is a radiolabel, such as ³²P, autoradiography is employed as the detection method. In one embodiment, the marker is a fluorophore, such as fluorescein, and the location of bound target DNA is determined by means of fluorescence spectroscopy. Automated equipment for use in fluorescence scanning of oligonucleotide probe arrays is available from Affymetrix, Inc. (Santa Clara, Calif.) and may be operated according to the manufacturer's instructions. Such equipment may be employed to determine the intensity of fluorescence at each predefined location on the array, thereby providing a measure of the amount of target DNA bound at each location. Such an assay would be able to indicate not only the absence and presence of the marker probe in the target, but also the quantitative amount as well.

In this manner, oligonucleotide probe kits of the present invention may be employed to examine the presence/absence (or relative amounts in case of mixtures) of polynucleotides in different biological samples, or products containing different materials, rapidly and in a cost-effective manner.

Another aspect of the present invention involves collections of a plurality of polynucleotides of the present invention. A collection of a plurality of the polynucleotides of the present invention, particularly the polynucleotides identified as SEQ ID NOS: 1-348, may be recorded and/or stored on a storage medium and subsequently accessed for purposes of analysis, comparison, etc. Suitable storage media include magnetic media such as magnetic diskettes, magnetic tapes, CD-ROM storage media, optical storage media, and the like. Suitable storage media and methods for recording and storing information, as well as accessing information such as polynucleotide sequences recorded on such media, are well known in the art. The polynucleotide information stored on the storage medium is preferably computer-readable and may be used for analysis and comparison of the polynucleotide information.

Another aspect of the present invention thus involves storage medium on which are recorded a collection of the polynucleotides of the present invention, particularly a collection of the polynucleotides identified as SEQ ID NOS: 1-348. According to one embodiment, the storage medium includes a collection of at least 20, preferably at least 50, more preferably at least 100, and most preferably at least 200 of the polynucleotides of the present invention, preferably the polynucleotides identified as SEQ ID NOS: 1-348, including variants of those polynucleotides.

In another aspect, the present invention provides genetic constructs comprising, in the 5′-3′ direction, a gene promoter sequence and an open reading frame coding for at least a functional portion of a polypeptide encoded by a polynucleotide of the present invention. In certain embodiments, the genetic constructs of the present invention also comprise a gene termination sequence. The open reading frame may be oriented in either a sense or antisense direction. Genetic constructs comprising a non-coding region of a gene coding for a polypeptide encoded by the above polynucleotides or a nucleotide sequence complementary to a non-coding region, together with a gene promoter sequence, are also provided. A terminator sequence may form part of this construct. Preferably, the gene promoter and termination sequences are functional in a host organism. More preferably, the gene promoter and termination sequences are common to those of the polynucleotide being introduced. The genetic construct may further include a marker for the identification of transformed cells.

Techniques for operatively linking the components of the genetic constructs are well known in the art and include the use of synthetic linkers containing one or more restriction endonuclease sites as described, for example, by Sambrook et al., in Molecular cloning: a laboratory manual, Cold Spring Harbor Laboratories Press: Cold Spring Harbor, N.Y., 1989. The genetic constructs of the present invention may be linked to a vector having at least one replication system, for example, E. coli, whereby after each manipulation, the resulting construct can be cloned and sequenced and the correctness of the manipulation determined.

Transgenic cells comprising the genetic constructs of the present invention are also provided by the present invention, together with organisms comprising such transgenic cells, products and progeny of such organisms. Techniques for stably incorporating genetic constructs into the genome of target organisms are well known in the art.

In one aspect, the present invention provides methods for using one or more of the inventive polypeptides or polynucleotides to treat disorders in a subject, preferably a mammal, including a human. In this aspect, the polypeptide or polynucleotide is generally present within a pharmaceutical composition or immunogenic composition. Pharmaceutical compositions may comprise one or more polypeptides, each of which may contain one or more of the above sequences (or variants thereof), and a physiologically acceptable carrier. immunogenic compositions may comprise one or more of the above polypeptides and an immunostimulant, such as an adjuvant.

Alternatively, a pharmaceutical or immunogenic composition of the present invention may contain a polynucleotide encoding one or more polypeptides as described above, such that the polypeptide is generated in situ. In such compositions, the polynucleotide may be present within any of a variety of delivery systems known to those of ordinary skill in the art, including nucleic acid expression systems, and bacterial and viral expression systems. Appropriate nucleic acid expression systems contain the necessary polynucleotide sequences for expression in a mammal (such as a suitable promoter and terminator signal). Bacterial delivery systems involve the administration of a bacterium (such as Bacillus Calmette-Guerin) that expresses an immunogenic portion of the polypeptide on its cell surface. In a preferred embodiment, the DNA may be introduced using a viral expression system (e.g., vaccinia or other poxvirus, retrovirus, or adenovirus), which may involve the use of a non-pathogenic, or defective, replication competent virus. Techniques for incorporating polynucleotides into such expression systems are well known in the art. The DNA may also be “naked,” as described, for example, in Ulmer et aL, Science 259:1745-1749, 1993; and reviewed by Cohen, Science 259:1691-1692, 1993. The uptake of naked DNA may be increased by coating the DNA onto biodegradable beads, which are efficiently transported into the cells.

Routes and frequency of administration, as well as dosage, will vary from individual to individual. In general, the pharmaceutical compositions and immunogenic compositions may be administered by injection (e.g., intradermal, intramuscular, intravenous, or subcutaneous); intranasally (e.g., by aspiration); or orally. In general, the amount of polypeptide present in a dose (or produced in situ by the DNA in a dose) ranges from about 1 pg to about 100 mg per kg of host, typically from about 10 pg to about 1 mg per kg of host, and preferably from about 100 pg to about 1 μg per kg of host. Suitable dose sizes will vary with the size of the mammal, but will typically range from about 0.1 ml to about 5 ml.

While any suitable carrier known to those of ordinary skill in the art may be employed in the pharmaceutical compositions of this invention, the preferred type of carrier will vary depending on the mode of administration. For parenteral administration, such as subcutaneous injection, the carrier preferably comprises water, saline, alcohol, a lipid, a wax, or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactic galactide) may also be employed as carriers for the pharmaceutical compositions of this invention. Suitable biodegradable microspheres are disclosed, for example, in U.S. Pat. Nos. 4,897,268 and 5,075,109.

Any of a variety of immunostimulants may be employed in the immunogenic compositions of this invention to non-specifically enhance the immune response. Most adjuvants contain a substance designed to protect the antigen from rapid catabolism, such as aluminum hydroxide or mineral oil, and a non-specific stimulator of immune responses, such as lipid A, Bordetella pertussis, or Mycobacterium tuberculosis. Suitable adjuvants are commercially available as, for example, Freund's Incomplete Adjuvant and Freund's Complete Adjuvant (Difco Laboratories, Detroit, Mich.), and Merck Adjuvant 65 (Merck and Company, Inc., Rahway, N.J.). Other suitable adjuvants include alum, biodegradable microspheres, monophosphoryl lipid A, and Quil A.

The polypeptides of the present invention may additionally be used in assays to determine biological activity, to raise antibodies, to isolate corresponding ligands or receptors, in assays to quantitatively determine levels of protein or cognate corresponding ligand or receptor, as anti-inflammatory agents, and in compositions for mammary glands, connective tissue and/or nerve tissue growth or regeneration.

The polynucleotides of the present invention may be used for expression in a transgenic animal, as disclosed in U.S. Pat. No. 5,714,345, which teaches the use of transgenic animals capable of expressing a desired protein prepared by introducing into an egg or embryo cell of an animal, an expression construct containing the sequence corresponding at least in part to a specific polynucleotide, which encodes the desired protein. In the same manner, the desired protein corresponding to a selected polynucleotide sequence of the present invention, may be employed in transgenic animals for the production of milk containing the desired protein, as disclosed in U.S. Pat. No. 5,849,992.

In addition, the regulatory sequences contained in the inventive polynucleotide sequences, or regulatory sequences isolated using the inventive sequences for genome screening and sequencing, as well known in the art, may be used in transgenic animals to direct the expression of a desired gene product according to the nature of the regulatory polynucleotide sequence, in a way similar to that taught in U.S. Pat. No. 5,850,000.

### EXAMPLE 1

### Isolation of cDNA Sequences from Bovine Mammary Gland cDNA Libraries

Bovine mammary gland cDNA expression libraries were constructed and screened as follows. mRNA was extracted from lactating bovine mammary tissue (late lactating, non-pregnant Jersey, 2 hours post-milking) using standard protocols. mRNA was precipitated with ethanol and the total RNA preparate was purified using a Poly(A) Quik mRNA Isolation Kit (Stratagene, La Jolla, Calif.). A cDNA expression library was constructed from the purified mRNA by reverse transcriptase synthesis followed by insertion of the resulting cDNA clones in Lambda ZAP using a ZAP Express cDNA Synthesis Kit (Stratagene), according to the manufacturer's protocol.

The resulting cDNAs were packaged using a Gigapack II Packaging Extract (Stratagene) employing 1 μl of sample DNA from the 5 μl ligation mix. Mass excision of the library was done using XL1-Blue MRF′ cells and XLOLR cells (Stratagene) with ExAssist helper phage (Stratagene). The excised phagemids were diluted with NZY broth (Gibco BRL, Gaithersburg, Md.) and plated out onto LB-kanamycin agar plates containing 5-bromo-4-chloro-3-indolyl-beta-D-galactoside (X-gal) and isopropylthio-beta-galactoside (IPTG).

Of the colonies plated and picked for DNA preparations, the large majority contained an insert suitable for sequencing. Positive colonies were cultured in NZY broth with kanamycin and cDNA was purified by means of REAL DNA minipreps (Qiagen, Venlo, The Netherlands). Agarose gel at 1% was used to screen sequencing templates for chromosomal contamination. Dye terminator sequences were prepared using a Biomek 2000 robot (Beckman Coulter Inc., Fullerton, Calif.) for liquid handling and DNA amplification using a 9700 PCR machine (Perkin Elmer/Applied Biosystems, Foster City, Calif.) according to the manufacturer's protocol.

The DNA sequences for positive clones were obtained using a Perkin Elmer/Applied Biosystems Division Prism 377 sequencer. cDNA clones were sequenced from the 5′ end. The sequences of the isolated polynucleotides are identified as SEQ ID NOS: 1-348. Amino acid sequences corresponding to the cDNA sequences of SEQ ID NO: 97, 103, 105, 114, 115, 121, 126, 129, 132, 143, 148, 151, 257, 275, 289, 294, 297, 306, 316, 332, 334 and 336 are provided in SEQ ID NO: 349-370, respectively.

BLASTN Polynucleotide Analysis

The isolated cDNA sequences were compared to sequences in the EMBL DNA database using the computer algorithm BLASTN. Comparisons of DNA sequences provided in SEQ ID NOS: 1-348 to sequences in the EMBL DNA database (using BLASTN) were made as of August, 2000, using Version 2.0.11 [Jan. 20, 2000], and the following Unix runing command: blastall −p blastn −d embldb −e 10 −G0 −E0 −r 1 −v 30 −b 30 −i queryseq −o.

The sequences of SEQ ID NOS: 1-74 and 152-239 were determined to have less than 50% identity, determined as described above, to sequences in the EMBL database using the computer algorithm BLASTN, as described above. The sequences of SEQ ID NOS: 75-95 and 240-274 were determined to have less than 75% identity, determined as described above, to sequences in the EMBL database using the computer algorithm BLASTN, as described above. The sequences of SEQ ID NOS: 96-116 and 275-299 were determined to have less than 90% identity, determined as described above, to sequences in the EMBL database using the computer algorithm BLASTN, as described above. Finally, the sequences of SEQ ID NOS: 117-141 and 300-334 were determined to have less than 98% identity, determined as described above, to sequences in the EMBL database using the computer algorithm BLASTN, as described above.

### EXAMPLE 2

### Expression of mRNA in Bovine Mammary Tissue

This example illustrates the determination of mRNA expression levels of mammary-gland specific sequences, isolated essentially as described in Example 1, in biological samples

RNA was extracted from mammary gland tissue obtained from a non-pregnant heifer, a pregnant cow and a lactating cow, as well as from bovine liver, forebrain and kidney, using TRIzol (Gibco BRL, Gaithersburg, Md.) following the manufacturer's protocol. Sets of the various total RNA samples were run on 1.2% agarose/formaldehyde gels, 5 μg/lane. Following transfer to nitrocellulose membranes, RNA was cross-linked with ultraviolet light.

DNA probes were prepared from bacterial clones transformed with cDNA corresponding to SEQ ID NOS: 200, 208, 227 and 273 by excision of the insert of the cDNA clone using EcoRI and Xhol restriction endonucleases, or by PCR amplification of the insert of the cDNA clone using T7 and T3 primers (Gibco BRL), or by using the entire cDNA clone. Probes were radiolabeled with α-P³²-dATP using Rediprime DNA labeling kits (Amersham Pharmacia Biotech, Uppsala, Sweden).

Blots were hybridized overnight with rotation at 65° C. in a buffer containing 10-20 ml of 500 mM NaH₂PO₄, 1 mM EDTA, 7% SDS, and then washed for 15 minutes at 65° C., first in 2× SSC/0.1% and then in 1× SSC/0.1% SDS. The blots were exposed to Kodak XAR X-ray film for appropriate times.

The insert of the cDNA clone corresponding to the polynucleotide sequence of SEQ ID NO: 200 hybridized strongly with a transcript of approximately 0.8 kb in the lactating mammary gland sample. Very weak hybridization to transcripts of a similar size was detected in the mammary samples from a non-pregnant heifer and a pregnant non-lactating cow. No transcripts could be detected in the other tissue samples.

The insert of the cDNA clone corresponding to the polynucleotide sequence of SEQ ID NO: 208 hybridized strongly to a transcript of approximately 1.2 kb in the lactating mammary gland. No transcripts were detected in the mammary samples from either a non-pregnant heifer or a pregnant non-lactating cow. Low levels of transcripts were detected in samples from brain and kidney but not from liver.

The insert of the cDNA clone corresponding to the polynucleotide sequence of SEQ ID NO: 227 hybridized strongly with a transcript of approximately 1.3 kb in the lactating mammary gland sample. In all other samples, a transcript of approximately 2.3 kb was detected with the expression levels greatest in the brain sample.

The insert of the cDNA clone corresponding to the polynucleotide sequence of SEQ ID NO: 273 hybridized strongly with a transcript of approximately 3.0 kb in the lactating mammary gland sample.

SEQ ID NOS: 1-370 are set out in the attached Sequence Listing. The codes for nucleotide sequences used in the attached Sequence Listing, including the symbol “n,” conform to WIPO Standard ST.25 (1998), Appendix 2, Table 1.

All references cited herein, including patent references and non-patent publications, are hereby incorporated by reference in their entireties.

While in the foregoing specification this invention has been described in relation to certain preferred embodiments, and many details have been set forth for purposes of illustration, it will be apparent to those skilled in the art that the invention is susceptible to additional embodiments and that certain of the details described herein may be varied considerably without departing from the basic principles of the invention.

We claim:

1. An isolated polynucleotide comprising a sequence selected from the group consisting of: (1) sequences recited in SEQ ID NOS: 1-348; (2) complements of the sequences recited in SEQ ID NOS: 1-348; (3) reverse complements of the sequences recited in SEQ ID NOS: 1-348; (4) reverse sequences of the sequences recited in SEQ ID NOS: 1-348; (5) sequences having at least a 99% probability of being the same as a sequence recited in (1)-(4) above as determined using computer algorithm BLASTN; (6) sequences having at least 75% identity to a nucleotide sequence recited in (1)-(4) above determined using computer algorithm BLASTN; (7) sequences having at least 90% identity to a nucleotide sequence recited in (1)-(4) above determined using computer algorithm BLASTN; (8) sequences having at least 95% identity to a nucleotide sequence recited in (1)-(4) above determined using computer algorithm BLASTN; (9) nucleotide sequences that hybridize to a sequence recited in (1)-(4) above under stringent hybridization conditions; (10) nucleotide sequences that are 200-mers of a sequence recited in (1)-(4) above; (11) nucleotide sequences that are 100-mers of a sequence recited in (1)-(4) above; (12) nucleotide sequences that are 40-mers of a sequence recited in (1)-(4) above; (13) nucleotide sequences that are 20-mers of a sequence recited in (1)-(4) above; and (14) nucleotide sequences that are degeneratively equivalent to a sequence recited in (1)-(4) above.

2. An oligonucleotide comprising at least 10 contiguous residues complementary to 10 contiguous residues of a nucleotide sequence recited in claim 1.

3. A genetic construct comprising an isolated polynucleotide of claim 1.

4. A host cell transformed with a genetic construct of claim 3.

5. An isolated polypeptide encoded by a polynucleotide of claim 1.

6. An isolated polypeptide comprising an amino acid sequence selected from the group consisting of: (a) sequences provided in SEQ ID NOS: 349-370; (b) sequences having at least a 99% probability of being the same as a sequence of SEQ ID NOS: 349-370, as determined using the computer algorithm BLASTP; (c) sequences having at least 75% identity to a sequence provided in SEQ ID NOS: 349-370, as determined using the computer algorithm BLASTP; (d) sequences having at least 90% identity to a sequence provided in SEQ ID NOS: 349-370 and 377-382, as determined using the computer algorithm BLASTP; and (e) sequences having at least 95% identity to a sequence provided in SEQ ID NOS: 349-370, as determined using the computer algorithm BLASTP.

7. An isolated polynucleotide encoding a polypeptide of claim 6.

8. An isolated polypeptide comprising at least a functional portion of an amino acid sequence selected from the group consisting of: (a) sequences provided in SEQ ID NOS: 349-370; (b) sequences having at least a 99% probability of being the same as a sequence of SEQ ID NOS: 349-370, as determined using the computer algorithm BLASTP; (c) sequences having at least 75% identity to a sequence provided in SEQ ID NOS: 349-370, as determined using the computer algorithm BLASTP; (d) sequences having at least 90% identity to a sequence provided in SEQ ID NOS: 349-370, as determined using the computer algorithm BLASTP; and (e) sequences having at least 95% identity to a sequence provided in SEQ ID NOS: 349-370, as determined using the computer algorithm BLASTP.

9. A composition comprising a polypeptide according to any one of claims 6 and 8 and at least one component selected from the group consisting of: physiologically acceptable carriers and immunostimulants.

10. A composition comprising a polynucleotide according to claim 1 and at least one component selected from the group consisting of: pharmaceutically acceptable carriers and immunostimulants.

11. A method for treating a disorder in a mammal comprising administering a composition according to claim 9.

12. A method for treating a disorder in a mammal comprising administering a composition according to claim 10.

13. A method for modifying mammary gland function or milk composition in an organism, comprising transforming the organism with a genetic construct according to claim 3.

14. A method for modifying mammary gland function, structure or composition in an organism, comprising administering a composition according to claim 9.

15. A method for detecting the presence of mammary gland tissue in a biological sample, comprising:
(a) contacting the biological sample with an oligonucleotide according to claim 2;
(b) detecting in the sample the presence of a polynucleotide that hybridizes to the oligonucleotide.

16. A diagnostic kit comprising at least one oligonucleotide according to claim 2.

17. A transgenic organism comprising a host cell according to claim 4.
